(19) United States
(12) Patent Application Publication
(10) Pub. No.: US 2022/0403429 A1
(43) Pub. Date: Dec. 22, 2022

(54) OPTOGENETIC CONTROL OF MICROBIAL CO-CULTURE POPULATIONS

(71) Applicant: The Trustees of Princeton University, Princeton, NJ (US)

(72) Inventors: Jose L. Avalos, Princeton, NJ (US); Makoto A. Lalwani, San Jose, CA (US); Mark P. Brynildsen, Pennington, NJ (US)

(73) Assignee: The Trustees of Princeton University, Princeton, NJ (US)

(21) Appl. No.: 17/844,835

(22) Filed: Jun. 21, 2022

Related U.S. Application Data
(60) Provisional application No. 63/212,762, filed on Jun. 21, 2021.

(51) Int. Cl. C12P 17/06; C12N 1/20; C12N 1/16; C12N 15/52; C12N 15/70; C12N 15/74

(52) U.S. Cl. CPC C12P 17/06; C12N 1/20; C12N 1/16; C12N 15/52; C12N 15/70; C12N 15/74; C12N 2529/10; C12N 2800/101; C12N 2830/001

(57) ABSTRACT

Microbial consortia exert great influence over the physiology of humans, animals, plants, and ecosystems. However, difficulty in controlling their composition and population dynamics have limited their application in medicine, agriculture, biotechnology, and the environment. The approach disclosed herein provides an effective method to dynamically control population compositions in microbial consortia, which we demonstrate in the context of co-culture fermentations for chemical production. Co-culture fermentations can improve chemical production from complex biosynthetic pathways over monocultures by distributing enzymes across multiple strains, thereby reducing metabolic burden, overcoming endogenous regulatory mechanisms, or exploiting natural traits of different microbial species. However, stabilizing and optimizing microbial sub-populations for maximal chemical production remains a major obstacle in the field. An optogenetic circuit, called OptoTA, is disclosed for regulating a toxin-antitoxin system, which enables tunability of, e.g., Escherichia coli growth using only blue light. With the disclosed system, one can control population ratios of co-cultures of, e.g., E. coli and Saccharomyces cerevisiae containing different metabolic modules of biosynthetic pathways. Results reveal that intermediate light duty cycles improve chemical production by establishing optimal co-culture populations.

### CROSS-REFERENCE TO RELATED APPLICATIONS

This application claims priority to U.S. Provisional Patent Application 63/212,762, filed Jun. 21, 2021, which is incorporated herein in its entirety.

### TECHNICAL FIELD

The present disclosure is drawn to optogenetically-controlled microorganisms useful for, e.g., fermentation processes.

### BACKGROUND

Metabolic engineering aims to rewire the metabolism of microorganisms to convert simple feedstocks into products of interest. Dynamic control, pathway compartmentalization, and adaptive laboratory evolution have helped to further improve microbial chemical production in engineered strains. However, overburdening an organism with genetic modifications can lead to growth defects and loss of productivity. Additionally, maximizing metabolic flux through a pathway of interest often requires bypassing endogenous regulatory mechanisms, functionally expressing heterologous proteins (including enzymes and transporters), or avoiding intermediate loss to competing pathways by deleting endogenous genes. When such interventions to enhance a particular step of a pathway undermine others, necessary compromises may lead to pathway inefficiencies. For example, when an enzyme important for one step of the pathway also competes in another step to make a byproduct, it may not help to overexpress or delete the enzyme. Another frequent challenge is the ability to functionally express some heterologous enzymes in organisms that otherwise have important advantages to produce a specific chemical. For example, the difficulty of expressing some eukaryotic enzymes in E. coli undermines the extraordinary ability of this organism to produce many precursors. These often-encountered challenges limit strain titers, yields, and productivities.

A solution that has been proposed to overcome these challenges is to separate and distribute metabolic roles among different members of a microbial consortium. Natural microbial consortia have been used, in some cases for millennia, to produce a wide variety of products, notably in the food and bulk chemical industries. Metabolic engineers have thus spent significant effort in engineering synthetic microbial consortia for chemical production. Co-culturing strains or organisms with specialized roles can improve productivity by reducing the total resources or genetic modifications required of each member. Furthermore, biosynthetic pathways can be split into submodules and be optimized based on the unique advantages of different species while avoiding endogenous regulation and competing pathways. Engineered microbial consortia have been applied to divide and optimize biosynthetic pathways within separate strains or species, leading to more efficient production of compounds such as muconic acid (by overcoming secretion of an intermediate), resveratrol (by avoiding downregulation of an intermediate pathway step), and rosmarinic acid (by suboptimization of pathway modules). They have also been utilized in consolidated bioprocessing approaches to more efficiently break down complex feedstocks, such as lignocellulosic biomass, into biofuels such as ethanol, butanol, and isobutanol, while avoiding the increases in production time, process complexity, and sterilization costs introduced by two-stage bioprocesses. Such studies demonstrate the benefits of combining the strengths of specialized strains or species to perform complex tasks.

Despite this progress made in engineering microbial consortia, maintaining population stability has remained an obstacle to their more widespread use and application in commercial processes. There is a natural propensity for the fastest growing member of a co-culture to eventually outcompete the others and establish a monoculture. The most common approach to address this problem is to adjust the initial inoculation sizes of each member to ensure they are all maintained throughout the fermentation. However, variations at inoculation (such as deviations in preculture age or measurement errors), amplified throughout fermentation, can lead to batch-to-batch inconsistencies. Furthermore, this strategy does not provide the opportunity to adjust microbial populations once the fermentation is initialized, which could help offset these variations. Clever solutions have been found to ensure all members of engineered co-cultures are maintained during fermentations by engineered neutrality or symbiosis: for example, by making strains reliant on different carbon or nutrient sources, modifying them to be co-dependent so that one species cannot grow without the other(s), or using quorum sensing to regulate growth via interpopulation communication. While these strategies have been largely successful at stabilizing members of engineered consortia, they still require careful tuning of parameters such as inoculation size and media components, which cannot be easily altered mid-fermentation. Furthermore, the population distributions established by these strategies are determined by the properties of each system and cannot be dynamically controlled to find, achieve, and maintain varying optimal population ratios to maximize chemical production.

A technique for controlling fermentation processes to produce e.g., a desired chemical of interest, that overcomes these limitations is therefore useful and desirable.

### BRIEF SUMMARY

In some embodiments, an engineered strain of a microorganism (such as an engineered strain of bacteria and/or yeast may be provided. The strain may include a genetic circuit configured to express a gene that enables growth when exposed to a light condition and express a gene that represses growth when not exposed to the light condition. The genetic circuit may include three polypeptide sequences: a first polypeptide sequence encoding a repressor gene, such as the λ phage repressor cI, under a first promoter, the first promoter being a light-responsive promoter; a second polypeptide sequence encoding either a toxin or an antitoxin gene, under a second promoter, such as a λ promoter; and a third polypeptide sequence encoding the other of the toxin or the antitoxin gene, under an additional first promoter.

In some embodiments, the genetic circuit may be configured to express a bacterial antitoxin (such as MazE) only when exposed to the light condition and a bacterial toxin (such as MazF) when not exposed to the light condition. In some embodiments, the bacteria may be a strain of E. coli. In some embodiments, the light condition is irradiation by a visible and/or infrared wavelength of light. In some embodiments, the genetic circuit may include circuits with known optical switches. In some embodiments, the genetic circuit may include pDawn and pDusk optogenetic circuits. In some embodiments, the first promoter is P_{FixK2}, and the second promoter is P_{R}.

In some embodiments, a method for regulating engineered microbial consortia may be provided. The method may include providing a plurality of microorganisms, where at least one microorganism includes a genetic circuit utilizing an optogenetic gene expression system to control an organism's growth rate using a specific wavelength of light or darkness. In some embodiments, the microorganism may be an embodiment of a strain of a microorganism (such as a bacteria or yeast) as disclosed herein. The method may include co-culturing the plurality of microorganisms in a light-controlled fermentation.

In some embodiments, a first organism of the plurality of microorganisms may include a biosynthetic pathway that produces an intermediate metabolite of a biosynthetic pathway of interest. In some embodiments, a second organism of the plurality of microorganisms may include a downstream biosynthetic pathway that converts the intermediate metabolite (such as an alcohol or aromatic amino acid) into a chemical of interest. In some embodiments, co-culturing the plurality of microorganisms in light-controlled fermentation comprises adjusting light conditions to control the co-culture populations and production of the chemical of interest (such as an ester or flavonoid).

In some embodiments, the method may include, in at least one of the plurality of microorganisms, transforming a strain with a first a plasmid, or vector that integrates into a chromosome, which contains the genetic circuit that uses an optogenetic gene expression system to control an organism's growth rate using a specific wavelength of light or darkness. In some embodiments, the method may include, in at least one of the plurality of microorganisms, transforming a strain with a second plasmid, or vector that integrates into a chromosome, which contains the biosynthetic pathway that produces the intermediate metabolite. In some embodiments, the method may include, in at least one of the plurality of microorganisms, transforming a strain with a third plasmid, or vector that integrates into a chromosome, which contains a biosynthetic pathway that converts an intermediate metabolite into the chemical of interest.

In some embodiments, the plurality of microorganisms may include one or more strains of E. coli, S. cerevisiae, P. putida, or a combination thereof.

In some embodiments, co-culturing the plurality of microorganisms in light-controlled fermentation may include illuminating the co-cultured fermentations with varying light schedules.

In some embodiments, the plurality of microorganisms comprises at least one bacterial species and at least one yeast species. In some embodiments, the bacteria-to-yeast ratio (BTYR) is BTYR<1. In some embodiments, BTYR=1. In some embodiments, BTYR>1.

In some embodiments, co-culturing may utilize a starting OD₆₀₀ of bacteria≤1×10⁻³. In some embodiments, co-culturing may utilize a starting OD₆₀₀ of bacteria>1×10⁻³. In some embodiments, co-culturing may utilize a starting OD₆₀₀ of yeast of≥0.1. In some embodiments, co-culturing may utilize a starting OD₆₀₀ of yeast of≤0.1.

### BRIEF DESCRIPTION OF DRAWINGS

FIG. 1 is an illustration of a genetic circuit.

FIG. 2 is an illustration of a system for producing a desired compound.

FIG. 3 is a flowchart of a method.

FIG. 4 is a graph showing final single cell E. coli/S. cerevisiae ratios of E. coli containing OptoTA (EMAL144) and wild-type yeast (yMAL272) grown for 24-h in co-cultures initiated from different inoculation sizes (shown as OD₆₀₀ values in table under the graph) in YNB-C medium and exposed to full light, 10 s ON/90 s OFF, or full darkness. Cells were counted with flow cytometry using mCherry and BFP to label EMAL144 and yMAL272, respectively.

FIG. 5 is a schematic illustration showing the isobutyl acetate biosynthetic pathway split into two modules, where module 1 (E. coli) produces isobutanol from glucose and is comprised of alsS from Bacillus subtilis, ilvC, ilvD, kivd from Lactococcus lactis, and adhA from L. lactis, and module 2 (S. cerevisiae) converts isobutanol and acetyl-CoA into isobutyl acetate and is comprised of ATF1 and feedback-insensitive mutant acs* from S. enterica (L641P). The acetate to make acetyl-CoA is derived from pyruvate produced from glycolysis, which is decarboxylated by Pdc1p and then oxidized by Ald6p.

FIG. 6 is a graph showing isobutyl acetate production from co-cultures of an E. coli strain containing OptoTA and module 1 (EMAL274) and a yeast strain containing module 2 (yMAL362), starting from different inoculation sizes (shown in table below graph) and grown for 48-h under different light conditions: full light, 10 s ON/90 s OFF, 1 s ON/99 s OFF, and full darkness, and production from co-cultures using a light-insensitive strain of E. coli (EMAL203), instead of EMAL274, grown in the dark, is shown as control. Statistics are derived using a two-sided t-test (**P<0.01); ns=not significant. All data are shown as mean values; error bars represent the s.d. of n=4 biologically independent samples.

FIG. 7 is a graph showing isobutanol production from EMAL274-yMAL362 co-cultures starting from different inoculation sizes (shown in table below graph) and incubated under different light conditions: full light, 10 s ON/90 s OFF, 1 s ON/99 s OFF, and full darkness. Production from EMAL203-yMAL362 co-cultures starting from different inoculation sizes and kept under ambient light is shown as a light-insensitive control.

FIG. 8 is a schematic illustration showing the naringenin biosynthetic pathway split into two modules: module 1 (E. coli) produces tyrosine from glucose and is comprised of tktA, ppsA, aroG_{D146N}, aroB, aroD, aroE, aroL, aroA, aroC, tyrA^{M53I, A354V}, and tyrB; module 2 (S. cerevisiae) converts tyrosine into naringenin and is comprised of FjTAL, At4CL2, HaCHS, PhCHI, and ACC1^{S650A, S1157A}. * denotes feedback-insensitive mutant enzymes.

FIG. 9 is a graph showing tyrosine production from monocultures of an E. coli strain containing module 1 (EMAL374) grown in M9 medium +2% glucose under full light, then induced with IPTG at different cell densities and fermented under 10 s ON/90 s OFF light for 48 h. UI denotes cultures not induced with IPTG.

FIG. 10 is a graph showing naringenin production from co-cultures of an E. coli strain containing OptoTA and module 1 (EMAL374) and a yeast strain containing module 2 (yMAL485), starting from different inoculation sizes (shown in table below graph) and grown for 48-h under different light conditions: full light, 10 s ON/90 s OFF, 1 s ON/99 s OFF, and full darkness. Production from co-cultures using a light-insensitive strain of E. coli (EMAL60), instead of EMAL374, grown in the dark is shown as control. Statistics are derived using a two-sided t-test. * P<0.05, *** P<0.001. All data are shown as mean values; error bars represent the s.d. of n=4 biologically independent samples.

### DETAILED DESCRIPTION

Disclosed herein is a technique for optogenetic control of microbial growth offering a promising solution for optimizing co-culture fermentations.

In some embodiments, an engineered strain of a microorganism may be provided. In some embodiments, the microorganism is a strain of bacteria, such as E. coli or P. putida. In some embodiments, the microorganism is a strain of yeast, such as S. cerevisiae.

The microorganism may include a genetic circuit configured to express a gene that enables growth when exposed to a light condition and express a gene that represses growth when not exposed to the light condition (e.g., kept in the dark, or exposed to a second light condition different from the first condition). For example, in some embodiments, the light condition may be blue light. When the organism is exposed to blue light, the organism may express genes enabling growth, and when the organism is kept in the dark or exposed to, e.g., red light, the organism may express genes that repress growth.

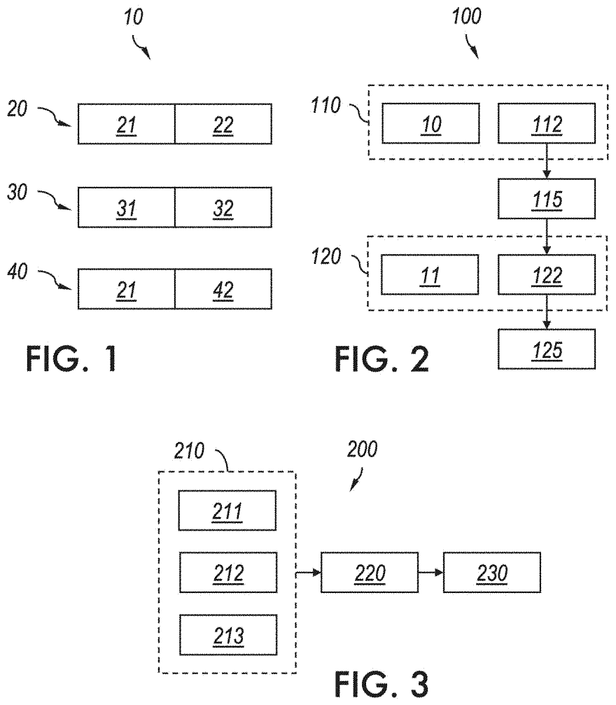

With reference to FIG. 1, a genetic circuit 10 may include three polypeptide sequences 20, 30, 40.

A first polypeptide sequence 20 may include a sequence 22 encoding a repressor, such as the λ phage repressor cI under a first promoter 21, the first promoter being a light-responsive promoter (including being a part of a light-inducible optical switch).

Light-responsive promoters and optical switches, and the wavelengths to which such promoters and switches respond are well-known in the art, and one skilled in the art would readily be able to incorporate any known light-responsive promoter (or switch including a promoter) into the disclosed microorganism. For example, the optical switch may utilize BLUF domains (such as bPAC which has a peak response at 441 nm), Phytochromes (such as BphP1-PpsR2 which has a peak response at 740-780 nm, or Phy-PIF which has a peak response at 660 nm), Cryptochromes (such as CIB1 having a peak response at 450 nm, OPTOSTIM having a peak response at around 488 nm), LOV domains (generally responsive in the 450-495 nm range, including iLID having a peak response at around 450 nm, pDusk having a peak response around 470 nm, or pDawn having a peak response around 470 nm), Dronpa-based switches (generally turning on around 400 nm, and turning off around 500 nm), or UVR8 domains (generally activating around 282 nm).

In some embodiments, the first promoter may be P_{FixK2}. In some embodiments, the genetic circuit comprises pDawn optogenetic circuits, pDusk optogenetic circuits, or a combination thereof. In some embodiments, the genetic circuit comprises only a pDawn optogenetic circuit. In some embodiments, the genetic circuit comprises only a pDusk optogenetic circuit.

In some embodiments, the light condition may be irradiation by a visible and/or infrared wavelength of light.

A second polypeptide sequence 30 may include a sequence 32 encoding either a toxin or an antitoxin, under a second promoter 31, the second promoter being a promoter controlled by the repressor encoded by sequence 22, such as λ promoter (e.g., a pL or pR promoter).

Toxin-Antitoxin systems are well-known in the art.

In some embodiments, the toxin and antitoxin are selected from known Type I toxin-antitoxin systems (which rely on the base-pairing of complementary antitoxin RNA with the toxin mRNA). Such systems include, e.g., (list shows toxins and then its antitoxin pair): hok and sok (identified in various gram-negative bacteria), fst and RNAII (identified in gram-positive bacteria), tisB and istR, dinQ and agrB, ldrD and rdlD (in Enterobacteriaceae),flmA and flmB, ibs and sib, txpA/brnT and ratA (in Bacillus subtilis), symE and symR, XCV2162 and ptaRNA1 (identified in Xanthomonas campestris), timP and timR (identified in Salmonella), aapA1 and isoA1 (identified in H. pylori), and sprA1 and sprA1as (disrupts S. aureus membranes and host erythrocytes).

In some embodiments, the toxin and antitoxin are selected from known Type II toxin-antitoxin systems (where a labile proteic antitoxin tightly binds and inhibits the activity of a stable toxin). Such systems include, e.g., (list shows toxins and then its antitoxin pair): ccdB and ccdA (identified in E. coli), parE and parD (identified in C. crescentus), mazF and mazE (identified in E. coli and in chromosomes of other bacteria), yafO and yafN (identified in E. coli), hicA and hicB (identified in achaea and bacteria), ζ and ε (identified in gram-positive bacteria), or ataT and ataR (identified in E. coli and Klebsiella spp.).

A third polypeptide sequence 40 may include a sequence 42 that encodes the other of the toxin or the antitoxin, under an additional first promoter 21. That is, if the second polypeptide sequence encodes for a toxin, the third polypeptide sequence should encode for the antitoxin, or if the second polypeptide sequence encodes for an antitoxin, the third polypeptide sequence should encode for the toxin.

In some embodiments, the genetic circuit may be configured to express a toxin (such as a bacteria toxin) only when exposed to the light condition and an antitoxin (such as a bacteria antitoxin) when not exposed to the light condition or when exposed to a second light condition different from the first light condition.

In some embodiments, the bacteria toxin may be MazF, and the bacteria antitoxin may be MazE.

Referring to FIG. 2, in some embodiments, a system 100 may be provided. The system may include a plurality of microorganisms 110, 120. In some embodiments, at least one of the plurality of microorganisms includes a genetic circuit 10, 11 utilizing an optogenetic gene expression system to control an organism's growth rate using a specific light condition. In some embodiments, all organisms in the system include such a genetic circuit, and preferably, a species-specific genetic circuit (e.g., a genetic circuit that is different from the genetic circuit in another species). In preferred embodiments, each species in the system has a genetic circuit where the light-responsive promoter in that circuit is responsive to a different light condition than the light-responsive promoters in other species. In that way, each species could be controlled by a unique light condition (e.g., different wavelengths of light or darkness). In some embodiments, one species of microorganism of the plurality of microorganisms does not include the genetic circuit disclosed herein, but all other microorganisms in the system do.

In some embodiments, a first organism 110 includes a genetic circuit 10 and a first biosynthetic pathway 112 that produces an intermediate metabolite 115. In some embodiments, a second organism 120 includes a genetic circuit 11 (which may be different from the genetic circuit 10 in first organism 110) and a second biosynthetic pathway 122 that is configured to convert the intermediate metabolite 115 into a chemical of interest 125. In some embodiments, the organisms are the same species. In some embodiments the organisms are different species of bacteria, yeast, or one organism is a species of bacteria, and the other organism is a species of yeast.

As will be understood, the intermediate metabolite may be any appropriate intermediate metabolite that the second biosynthetic pathway can use to convert to a chemical of interest. In some embodiments, the intermediate metabolite may be, e.g., an alcohol or aromatic amino acid. In some embodiments, the chemical of interest may be, e.g., an alcohol, a sugar, an ester, a flavonoid, etc.

It is understood that complete pathways to produce a desired compound of interest are well-understood in the art. In the present disclosed approach, these known complete pathways are preferably separated into at least two parts or modules—one module for use in a first microorganism and another module for use in a second, different microorganism, and so on—with only routine experimentation required. For example, in some embodiments, the pathway may be broken into three parts, each part being present in a different microorganism. In some embodiments, the first module is not naturally present in the first microorganism, and the second module is not naturally present in the second, different microorganism.

Referring to FIG. 3, in some embodiments, a method 200 for regulating engineered microbial consortia may be provided.

In some embodiments, the method may include preparing 210 one or more microorganisms of a plurality of organisms.

In some embodiments, preparing microorganisms includes, in at least one of the plurality of microorganisms, providing 211 a genetic circuit to the organism by, e.g., either transforming a strain with a first plasmid containing the genetic circuit, or with a first vector that integrates the genetic circuit into a chromosome, where the genetic circuit uses an optogenetic gene expression system to control an organism's growth rate using a specific wavelength of light.

In some embodiments, preparing microorganisms includes, in at least one of the plurality of microorganisms, providing 212 a biosynthetic pathway to the organism for producing the intermediate metabolite by, e.g., either transforming a strain with a second plasmid containing the biosynthetic pathway, or with a second vector that integrates the biosynthetic pathway into a chromosome.

In some embodiments, preparing microorganisms includes, in at least one of the plurality of microorganisms, providing 213 a biosynthetic pathway to the organism for converting the intermediate metabolite into a chemical of interest by, e.g., either transforming a strain with a third plasmid containing the biosynthetic pathway, or with a third vector that integrates the biosynthetic pathway into a chromosome. As will be understood, the organism used to produce the intermediate metabolite should be a different strain/species than the organism used to convert the intermediate metabolite to a chemical of interest.

In some embodiments, a complete biosynthetic pathway may utilize only two strains of microorganisms—one to create the intermediate, and one to create the chemical of interest. In some embodiments, a complete biosynthetic pathway may utilize multiple microorganisms where each produces an intermediate metabolite, and only a final organism makes the chemical of interest. For example, a first organism configured to create a first intermediate, a second organism that uses the first intermediate and produces a second intermediate, etc., until the final microorganism takes a final intermediate and converts it to a chemical of interest.

The method may include providing a plurality of microorganisms 220. In some embodiments, the plurality of microorganisms may include one or more strains of E. coli, S. cerevisiae, P. putida, or a combination thereof.

In some embodiments, the plurality of microorganisms comprises at least one bacteria species and at least one yeast species. In some embodiments, the bacteria-to-yeast ratio is less than 1. In some embodiments, the bacteria-to-yeast ratio is equal to 1. In some embodiments, the bacteria-to-yeast ratio is greater than 1.

The microorganisms may each be an embodiment of a microorganism as disclosed herein. In some embodiments, at least one of the plurality of microorganisms includes a genetic circuit utilizing an optogenetic gene expression system to control an organism's growth rate using a specific wavelength of light.

In some embodiments, at least a first organism of the plurality of microorganisms includes a biosynthetic pathway that produces an intermediate metabolite and at least a second organism of the plurality of microorganisms includes a biosynthetic pathway that converts the intermediate metabolite into a chemical of interest.

The method may include co-culturing 230 the plurality of microorganisms in a light-controlled fermentation. In some embodiments, co-culturing the plurality of microorganisms in light-controlled fermentations includes adjusting light conditions to control the co-culture population composition and production of the chemical of interest. In some embodiments, controlling production includes adjusting which wavelengths of light (if any) the plurality of microorganisms are exposed to at any given point in time of the fermentation, to adjust to co-culture population composition and increase the amount of chemical of interest produced as compared to fermenting under static light conditions.

In some embodiments, co-culturing the plurality of microorganisms in light-controlled fermentation comprises illuminating the co-cultured fermentations with varying light schedules. For example, in some embodiments, periods of irradiation and darkness may be used, where each period may vary during the fermentation process.

As will be understood, the intermediate metabolite may be any appropriate intermediate metabolite that the second biosynthetic pathway can use to convert to a chemical of interest. In some embodiments, the intermediate metabolite may be, e.g., an alcohol or aromatic amino acid. In some embodiments, the chemical of interest may be, e.g., an alcohol, a sugar, an ester, a flavonoid, etc.

In some embodiments, co-culturing may utilize a starting OD₆₀₀ of bacteria≤5. In some embodiments, co-culturing may utilize a starting OD₆₀₀ of bacteria>1×10⁻³. In some embodiments, co-culturing may utilize a starting OD₆₀₀ of yeast of≥0.1. In some embodiments, co-culturing may utilize a starting OD₆₀₀ of yeast of≤10.

### Example 1

To establish optogenetic control over E. coli, one can apply the pDusk and pDawn systems to control the MazF/MazE Toxin/Anti-toxin system using darkness and blue light. Briefly, pDusk uses the synthetic two-component YF1-FixJ system to activate gene expression in darkness, while pDawn reverses the signal of pDusk using the cI repressor to activate expression in blue light. The pDusk/pDawn system is comprised of a chimeric light-responsive and reversible histidine kinase YF1, its cognate response regulator FixJ, and the FixJ-activated P_{FixK2} promoter. In pDusk, YF1 phosphorylates FixJ in the dark, which allows it to activate P_{FixK2} to directly transcribe genes of interest in the absence of light. The pDawn circuit inverts the response of pDusk to activate gene expression in blue light. In pDawn, P_{FixK2} drives expression of the λ phage repressor cI, which suppresses transcription from its cognate promoter P_{R}. Under blue light (˜470 nm), YF1 dephosphorylates FixJ, preventing P_{FixK2}-mediated transcription of cI, thus allowing transcription of genes of interest controlled by P_{R}.

Using these systems, a new optogenetic circuit was created, which is referred to herein as OptoTA, to control bacterial growth with light. OptoTA expresses mazF using pDusk (P_{FixK2}) and mazE using pDawn (P_{R}), such that MazE-mediated inhibition of MazF enables growth in blue light, while repression of mazE and expression of mazF prevents growth in darkness.

OptoTA was characterized in E. coli strains derived from MO001 (MG1655 1acI^{q} ΔlacZYA::P_{T5}_mCherry). The endogenous copies of mazE and mazF were simultaneously knocked out of MO001 and the kanamycin resistance marker removed through FLP-FRT recombination to make EMAL142. A plasmid containing pDawn controlling mazE and pDusk controlling mazF (OptoTA, pMAL511) was used to transform electrocompetent strain EMAL142 to make EMAL144. As a control, an empty plasmid containing pDusk was used to transform strain EMAL142 to make EMAL221.

To evaluate light dependence of E. coli growth in different media, 1 mL overnight cultures of EMAL221 were inoculated into M9 medium+kanamycin+2% glucose, as well as EMAL144 into M9 medium+kanamycin+2% glucose, glycerol, or xylose, or LB+kanamycin, under constant blue light. The next day, the cultures were back-diluted into the same media to OD₆₀₀=0.01 in 1 mL into 24-well plates and grew the cultures for 24 h (or 12 h for LB) under blue light or in the dark, after which point OD₆₀₀ measurements were taken.

When grown in M9 media containing glucose as a carbon source, a ΔmazEF strain containing OptoTA shows 19-fold higher growth under full blue light compared to darkness, while reaching 77% of the cell density of a control strain that is not light-dependent, suggesting that MazE-mediated repression of MazF is either incomplete or that MazEF production carries some burden. The difference between light and dark (27-fold) is even higher in xylose. Additionally, growth in darkness is lower (OD₆₀₀<0.25 after 24 h at 30° C.) under different carbon sources such as glycerol and xylose than in glucose, but higher (OD₆₀₀=0.57 after 12 h) in the richer lysogeny broth (LB), consistent with previous observations that MazF-mediated toxicity is media-dependent. These results show that OptoTA can regulate E. coli growth in several types of media commonly used for biotechnological applications.

To determine whether light dosage can be used to tune growth rate using OptoTA, one can perform growth curve experiments under different light duty cycles, chosen based on the rapid time scales of YF1 phosphorylation of FixJ (<30 s) and activation times of pDusk and pDawn (<10 min).

To evaluate kinetics of E. coli growth using OptoTA under different light conditions, 1 mL overnight cultures of EMAL144 and EMAL221 were inoculated into M9 medium+kanamycin+2% glucose media under blue light. The next day, the cultures were back-diluted to OD₆₀₀=0.01 in 1 mL into 24-well plates and grew the cultures until steady state was reached. Light conditions tested for EMAL144 were full blue light; pulses of 10 s ON/90 s OFF, 1 s ON/99 s OFF; and full darkness (plates wrapped in aluminum foil). OD₆₀₀ measurements were taken every 3-5 h.

Intermediate duty cycles (10 s or 1 s of light per 100 s) result in intermediate growth profiles between full light and darkness, showing that growth rate is titratable by varying light exposure. A 10% light dose (10 s ON/90 s OFF) of light recovers 72% of the growth rate observed in full light (0.17 vs 0.23 h⁻¹), though less light exposure results in a ˜1-h increase in lag phase. This strain containing OptoTA, however, shows a 20% decrease in growth rate under full light compared to a wild-type control (0.23 vs 0.29 h⁻³). Nevertheless, the ability of OptoTA to tune bacterial growth provides the capability needed to dynamically control populations in co-cultures with other species.

### Example 2 (Optogenetic Control of a Consortium of E. coli and S. cerevisiae)

The optogenetic regulation of E. coli growth attainable with OptoTA is sufficient to control populations in co-cultures with other organisms with lower growth rates that would otherwise be outcompeted in media favorable to E. coli (which is the case for common culturing conditions), such as the model yeast S. cerevisiae. However, S. cerevisiae does not grow on M9 medium (commonly used in E. coli cultures), while E. coli does not grow on synthetic complete (SC) medium (commonly used in S. cerevisiae cultures). To find a defined minimal medium that would support both E. coli and S. cerevisiae cultivation, while still favoring E. coli, combinations of commonly used bacterial buffers—M9 and K3—were tested with two commonly used yeast media—yeast nitrogen base (YNB) and SC.

### Plasmid and Strain Construction

Plasmids were cloned into E. coli strain DH5α made chemically competent using the Inoue method. Transformants were inoculated on LB agar plates with appropriate antibiotics: 100 μg/mL ampicillin, 100 μg/mL carbenicillin, 50 μg/mL kanamycin, or 34 μg/mL chloramphenicol. Epoch DNA Miniprep, Omega E.Z.N.A. Gel Extraction, and Omega E.Z.N.A. Cycle Pure kits were used to extract and purify plasmids and DNA fragments. Genes were amplified from bacterial or yeast genomic DNA or lab plasmids. Backbones and inserts were either digested using restriction enzymes purchased from NEB or PCR amplified using CloneAmp HiFi PCR premix from Takara Bio. Primers were ordered from Integrated DNA Technologies (Coralville, Iowa). Gibson isothermal assembly was performed based on utilizing protocols known in the art. For S. cerevisiae constructs, promoter-gene-terminator sequences were cloned into standardized vector series (pJLA vectors) as known in the art. All plasmids were verified using Sanger sequencing from Genewiz. Tandem repeats were avoided to prevent recombination after transformation, and thus do not observe instability of strains or plasmids.

Plasmids pDusk and pDawn were obtained as a gift (Addgene #43795 and #43796, respectively).

### Bacterial and Yeast Transformations

Deletion of endogenous mazEF in E. coli was performed using the Datsenko-Wanner method. Cells were made electrocompetent and electroporated as known in the art. FRT-flanked resistance markers were cured using FLP recombinase from pCP20. Gene deletions were genotyped by sequencing PCR products amplified from purified genomic DNA using primers flanking the region of deletion. Yeast transformations were carried out using standard lithium acetate protocols as known in the art. Gene constructs derived from pYZ162 or pYZ23 vectors were linearized with PmeI and genomically integrated into the LEU2 locus or δ-sites (YARCdelta5), respectively. Zeocin (purchased from Thermo Fisher Scientific) was used at a concentration of 1200 μg/mL to select for δ-integration.

A strain of E. coli containing OptoTA shows differences in growth between blue light and darkness in each of these hybrid media, though growth in darkness is significantly increased in buffered SC media (OD₆₀₀>2 after 14 h at 30° C.), suggesting that the toxicity of MazF is attenuated by supplementation of nutrients such as amino acids, probably due to reduced degradation of MazE by Lon protease, which occurs under conditions of nutrient starvation. YNB media supplemented with glucose and M9 buffer shows the best optogenetic control over E. coli growth (5-fold difference in OD₆₀₀) while still supporting yeast growth, albeit with a longer lag phase. The experiments continued using this media formulation, which is referred to as YNB-Consortia (YNB-C).

To test the viability of E. coli and S. cerevisiae on SC and M9 2% agar plates, 1 mL overnight cultures of EMAL144 were inoculated into M9 medium+kanamycin+2% glucose under constant blue light and CEN.PK2-1C into SC+2% glucose. The next day, both strains were streaked out onto an M9+kanamycin+2% glucose agar plate, as well as an SC+2% glucose agar plate, and both plates were incubated under blue light at 30° C. for 36 h.

To screen for media formulations in which both E. coli and S. cerevisiae could grow and in which OptoTA would still be effective, 1 mL overnight cultures of EMAL144 were inoculated into M9 medium+kanamycin+2% glucose under constant blue light. The next day, the cultures were back-diluted to OD₆₀₀=0.01 in 1 mL into 24-well plates into the following media: YNB+M9 salts buffer (YNB-C); YNB+K3 salts buffer; SC+M9 salts buffer; and SC+K3 salts buffer. All media contained 2% glucose and kanamycin. The cultures were then grown for 14 h under blue light or in the dark, after which OD₆₀₀ measurements were taken.

To characterize the growth of S. cerevisiae in YNB-C media, 1 mL overnight cultures of CEN.PK2-1C were inoculated into YNB+2% glucose or YNB-C+2% glucose. The next day, the cultures were back-diluted into the same media to OD₆₀₀=0.1 in 1 mL into 24-well plates and the cultures grown until steady state.

To characterize potential toxic effects of light toward S. cerevisiae, 1 mL overnight cultures of CEN.PK2-1C we inoculated into SC+2% glucose. The next day, the cultures were back-diluted into the same media to OD₆₀₀=0.01 in 1 mL into 24-well plates under blue light and darkness and the cultures were grown until steady state.

To characterize the growth dynamics of E. coli and S. cerevisiae in a consortium, one can examine how light control can create divergent population ratios from the same initial inoculation. To do so, co-cultures were inoculated to different initial cell densities for each species in YNB-C medium and then grown under different light conditions (e.g., under blue light, darkness, etc.) Because OD₆₀₀ cannot be used to distinguish population sizes in co-cultures due to the differing optical properties of each species, cell counts were quantified using flow cytometry with counting beads, distinguishing bacterial and yeast populations by light scattering properties and expression of distinct fluorophores—mCherry for E. coli and BFP for S. cerevisiae.

To evaluate light-dependence of strains grown in consortium, constitutively expressed TagBFP (pMAL653) was integrated into the LEU2 locus of CEN.PK2-1C to create yMAL272. Overnight cultures of EMAL221, EMAL144 (both of which express mCherry when induced with IPTG), and yMAL272 were inoculated separately in YNB-C+2% glucose+kanamycin media (under full blue light for EMAL144). The next day, these overnight cultures were used to inoculate 150 μL of fresh media (same medium as the overnights) +1 mM IPTG to induce mCherry expression, in 96-well plates to calculated OD₆₀₀ values of 10⁻⁵−10⁻³ for bacteria and 0.1-1 for yeast. The co-cultures were then grown at 30° C. under different blue light conditions, including full light; 10 s ON/90 s OFF pulses; and full darkness (plates wrapped in aluminum foil). After 12 h, 5 μL of each culture was diluted into 95 μL of ice-cold PBS containing the manufacturer's recommended concentration (500 μL of beads per 10 mL) of fluorescent counting beads for flow cytometry analysis.

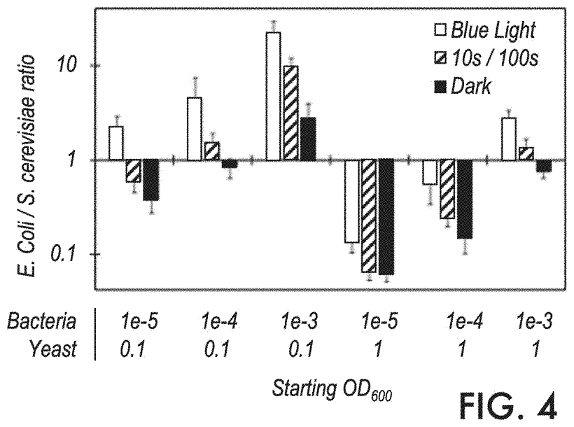

When using a strain of E. coli that is not light-regulated, the population ratios after 12 h of co-culture at 30° C. greatly favor E. coli in most inoculation ratios. A final bacteria-to-yeast ratio <1 is only achieved when inoculating a very small amount of E. coli (OD₆₀₀=1×10⁻⁵, or ˜1×10⁴ cells/mL) and a much larger amount of S. cerevisiae (OD₆₀₀=1, or ˜1×10⁷ cells/mL), reflecting the need to give yeast a considerable advantage in initial inoculation to compensate for its slower growth in YNB-C medium. Such large disparities in inoculation size could reduce the efficiency of co-culture fermentations, in which the relative abundances of biosynthetic submodules must be carefully balanced to maximize productivity. In contrast, when controlling E. coli growth with light using OptoTA, it becomes possible to reach bacteria-to-yeast ratios of <1 even at much lower yeast (OD₆₀₀=0.1) or higher bacterial (OD₆₀₀=1×10⁻³) inoculations (see FIG. 4). Such inoculations are more balanced and more closely represent ratios previously used in E. coli-S. cerevisiae co-cultures (cell ratios ˜1:100-100:1). Thus, it is possible to obtain several final population ratios from the same initial inoculation, or to reach similar ratios from different initial inoculations, simply by changing light duty cycle. Thus, the light control enabled by OptoTA provides an additional degree of freedom that augments adjustment of inoculation sizes to control microbial populations in co-culture fermentations.

### Example 3 (Production of Isobutyl Acetate Using an E. coli-S. cerevisiae Consortium)

Precise control over microbial co-cultures holds promise for applications in metabolic engineering, in which biosynthetic pathways can be divided into separate modules among different species to make products of interest. To test this, a division of labor strategy was devised to produce isobutyl acetate, a chemical solvent, fragrance, and potential biofuel that is derived from isobutanol and acetyl-CoA.

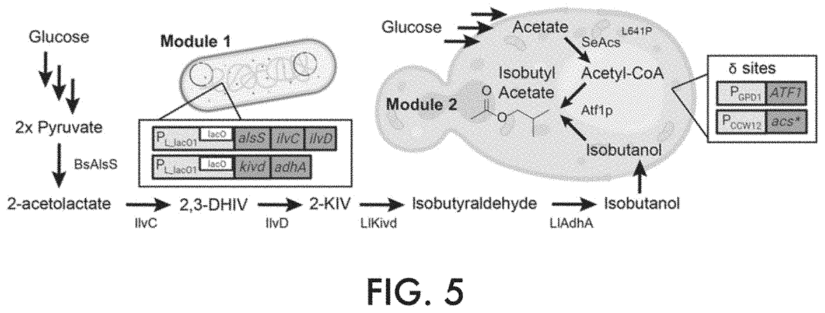

The biosynthetic pathway for isobutyl acetate was divided into two modules: an E. coli module that produces isobutanol, followed by an S. cerevisiae module that combines isobutanol with acetyl-CoA to produce isobutyl acetate (see FIG. 5). Isobutanol and acetyl-CoA are both derived from pyruvate in opposing pathways, so there is an inherent competition in the biosynthesis of these two precursors that could be resolved using two specialized strains in co-culture. A light-controlled isobutanol-producing strain of E. coli was engineered by transforming a ΔmazEF strain with plasmids containing OptoTA and the isobutanol biosynthetic pathway. This strain (EMAL274) secretes up to 3.9±0.3 g/L isobutanol into the culture supernatant when grown in M9 media, with production peaking when IPTG induction occurs at intermediate cell densities (0.2≤ρ≤0.9, where ρ represents the cell density at induction, measured as OD₆₀₀).

A strain of S. cerevisiae with increased acetyl-CoA production was then engineered that could assimilate isobutanol from the culture supernatant and convert it into isobutyl acetate. To achieve this, multiple copies of a feedback-insensitive acetyl-CoA synthetase from Salmonella enterica (acs L641P) and the yeast alcohol acetyltransferase (ATF1) was integrated into the δ-sites of CEN.PK2-1C. The resulting strain (yMAL362) produces up to 90±10 mg/L isobutyl acetate when grown to various cell densities and then resuspended in SC media containing 1 g/L isobutanol, with production saturating around ρ≥6.6.

These results show that our bacterial strain can produce and export isobutanol while our yeast strain can import it and convert it to isobutyl acetate, offering the potential for each module to be optimized independently to balance demand for the acetyl-CoA precursor while completing a complex production pathway.

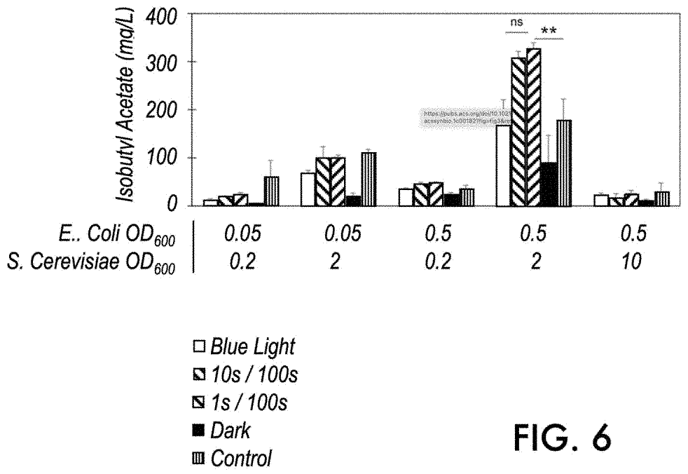

To determine if optogenetics can be used to improve chemical production from microbial consortia, the bacterial and yeast modules were combined to produce isobutyl acetate in light-controlled fermentations. Co-cultures of EMAL274 and yMAL362 were grown at 30° C. for 48 h under various light conditions and initial inoculum sizes, using 1 mM IPTG to induce isobutanol production at the time of co-inoculation. It was found that isobutyl acetate production depends greatly not only on the inoculation but also on the light duty cycles applied to the co-cultures (see FIG. 6).

The highest titer of isobutyl acetate (330±10 mg/L) was obtained using intermediate inoculation sizes of bacteria (OD₆₀₀=0.5) and yeast (OD₆₀₀=2), as well as a 1% duty cycle of light (1 s ON/100 s). This isobutyl acetate production is 6.9-fold or 12.3-fold higher than if the co-culture is inoculated with less yeast (OD₆₀₀=0.2, 48±3 mg/L) or more yeast (OD₆₀₀=10, 27±6 mg/L), respectively (while keeping the inoculation size of bacteria constant), emphasizing the influence of initial inoculation on fermentation productivity.

However, within the most productive inoculation, light control of the consortia population during the fermentation can significantly improve production by 71% (from 190±30 mg/L) relative to conditions that would favor bacterial growth (under continuous light), and 3.6-fold higher (from 90±60 mg/L) relative to conditions that would favor yeast growth (under darkness). These results demonstrate the benefit of adjusting microbial populations from productive inoculums during co-culture fermentation, which we achieve with light control of bacterial cell growth using OptoTA.

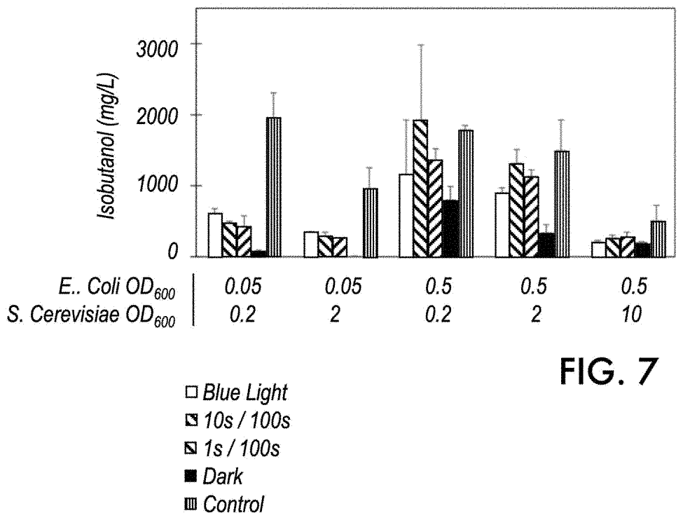

Light duty cycles in optogenetically controlled co-cultures can also influence the levels of intermediates relayed between strains, shedding light on the system's dynamics. Production of the intermediate, isobutanol, increases with higher light exposure at lower bacterial inoculations, but is maximized by intermediate light exposures at higher inoculations (see FIG. 7).

These results suggest that intermediate light doses help tune bacterial growth to maximize production of isobutanol without outcompeting yeast, thereby leading to improved isobutyl acetate production. This is supported by the observation that under the most productive conditions found, isobutyl acetate production is 83% higher using a light-regulated bacterial strain relative to using a light-insensitive strain (180±40 mg/L), even though isobutanol production is comparable. The levels of isobutanol ultimately reflect the bacterial population and reveal that yeast-catalyzed esterification is limiting, probably because of limited acetyl-CoA supply, which would need to be further improved to increase conversion to isobutyl acetate.

Serial dilution analysis of consortia populations shows that bacterial growth is still light-controlled under fermentation conditions, while yeast growth is slightly more efficient under lower light dosage, likely due to less competition from E. coli.

There is no apparent contribution from phototoxicity, as yeast growth is the same under both full light and darkness. Bacteria-only controls solely produce isobutanol, while yeast-only controls produce neither isobutanol nor isobutyl acetate, confirming that isobutyl acetate is produced only by combining the two strains in co-cultures.

### Example 4 (Production of Naringenin Using an E. coli-S. cerevisiae Consortium)

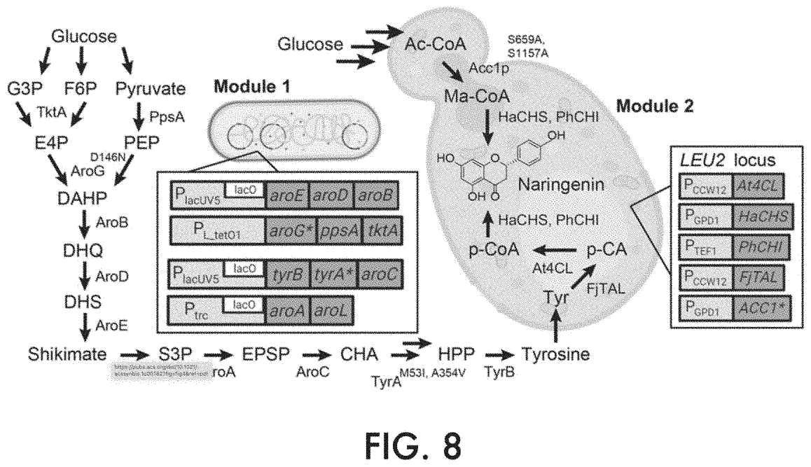

To demonstrate that light-based control of microbial consortia can be extended to other metabolic pathways, a co-culture production strategy was developed for naringenin, a natural product with potential therapeutic properties. This long metabolic pathway, which starting from increasing aromatic amino acid biosynthesis comprises 16 different enzymes, can load a single strain with excessive metabolic burden. Thus, taking advantage of E. coli's sound ability to produce aromatic amino acids, and aiming to bypass the strong endogenous regulation of aromatic amino acid biosynthesis in yeast, the naringenin production pathway was split into an E. coli module that produces and exports tyrosine followed by an S. cerevisiae module that intakes tyrosine and combines it with malonyl-CoA to produce naringenin (see FIG. 8).

To engineer a light-responsive E. coli strain to produce tyrosine, a ΔmazEF strain was transformed with plasmids encoding for OptoTA and enzymes to enhance tyrosine biosynthesis.

To make a light-sensitive strain of E. coli that produces tyrosine, we transformed electrocompetent EMAL142 with pMAL511 (containing OptoTA), and plasmids pS4 and pY3, containing 11 genes combined that convert glucose into tyrosine: transketolase (tktA); phosphoenolpyruvate synthase (ppsA); feedback-insensitive DAHP synthase (aroG^{D146N}); DHQ synthase (aroB); DHQ dehydratase (aroD); shikimate dehydrogenase (aroE); shikimate kinase II (aroL); EPSP synthase (aroA); chorismate synthase (aroC); feedback-insensitive chorismate mutase/prephenate dehydrogenase (tyrA^{M531, A354V}); and tyrosine aminotransferase (tyrB). The resulting strain, EMAL374, was plated onto LB+kanamycin+ampicillin+chloramphenicol agar plates and incubated under full light. As a light-insensitive control, electrocompetent MG1655 was transformed with pS4 and pY3 to create EMAL60, which was plated onto LB+ampicillin +chloramphenicol agar plates. Eight colonies were then used to inoculate 1 mL of M9 medium+2% glucose+carbenicillin+chloramphenicol media and grown overnight (under blue light and+kanamycin for EMAL374). The next day, the same procedures were followed as for isobutanol to identify the highest producing colony; also sealing the plates, but with a single hole poked to allow for limited aeration. After 48 h of fermentation under ambient light for EMAL60 or 10% light for EMAL374, samples were prepared for HPLC analysis.

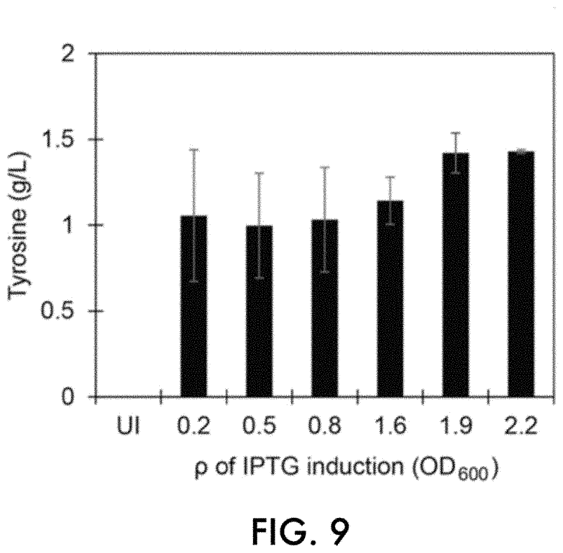

To find the optimal cell density at which tyrosine production is induced with IPTG we followed the same procedure was followed as described above for isobutanol production, except using both carbenicillin and chloramphenicol for selection (with blue light and kanamycin added for EMAL374). This strain (EMAL374) exports up to 1.4±0.1 g/L tyrosine into the supernatant when grown in M9 media, with production peaking when inducing with IPTG at relatively high cell densities (ρ>1.9) (see FIG. 9).

Next, to produce a S. cerevisiae strain that produces naringenin from imported tyrosine, the naringenin biosynthetic pathway was integrated, as well as feedback-insensitive acetyl-CoA carboxylase, into CEN.PK2-1C.

To construct a S. cerevisiae strain that converts tyrosine into naringenin, we integrated tyrosine ammonia-lyase from Flavobacterium johnsoniaeu (FjTAL), (50) 4-coumarate-CoA ligase 2 from Arabidopsis thaliana(At4CL2), chalcone synthase from Hypericum androsaemum (HaCHS), chalcone isomerase from Petunia hybrida (PhCHI), and feedback-insensitive acetyl-CoA carboxylase (ACC1^{S650A, S1157A}) into the LEU2 locus of CEN.PK2-1C (linearized pMAL959), creating yMAL485. Transformants were plated on SC—Leu+2% glucose agar. Eight colonies were then used to inoculate 1 mL overnight cultures of SC+2% glucose media. The next day, the same procedures were followed as for isobutyl acetate, using 1 g/L tyrosine in the media and sealed plates with a single hole poked on the tape. After 48 h of fermentation, samples were prepared for HPLC-MS analysis as described below. The highest producing colony was selected for subsequent analysis.

To find the optimal density at which to feed tyrosine to maximize naringenin production, the same procedure as above for isobutyl acetate production was followed by feeding tyrosine instead of isobutanol. Naringenin production from this strain (yMAL485) in SC media shows no dependence on the cell density at which tyrosine is fed, reaching 12-14 mg/L from 1 g/L tyrosine

Co-cultures of the tyrosine and naringenin modules were then tested as to whether they can be controlled with light to improve naringenin production.

To produce naringenin using an E. coli-S. cerevisiae consortium, 50 mL overnight cultures of EMAL60 or EMAL374 were inoculated into M9 medium+2% glucose+carbenicillin +chloramphenicol media (under blue light and+kanamycin for EMAL374), as well as yMAL485 into SC+2% glucose media. The next day, these cultures were centrifuged at 3700 rpm for 10 min, resuspended in ESC+4% glucose+carbenicillin+chloramphenicol media (+kanamycin for EMAL374), and back-diluted in 1 mL to the following OD₆₀₀ values: 0.5 or 5 for EMAL60 and EMAL374; and 1 or 10 for yMAL485. The plates were then sealed with a single hole poked to allow for limited aeration, and the co-cultures were fermented for 48 h under the following light conditions: full blue light, 10 s ON/90 s OFF, 1 s ON/99 s OFF, or full darkness (plates wrapped in aluminum foil). Co-cultures containing EMAL60 were grown in the dark. After 48 h, samples were centrifuged and collected for HPLC-MS analysis.

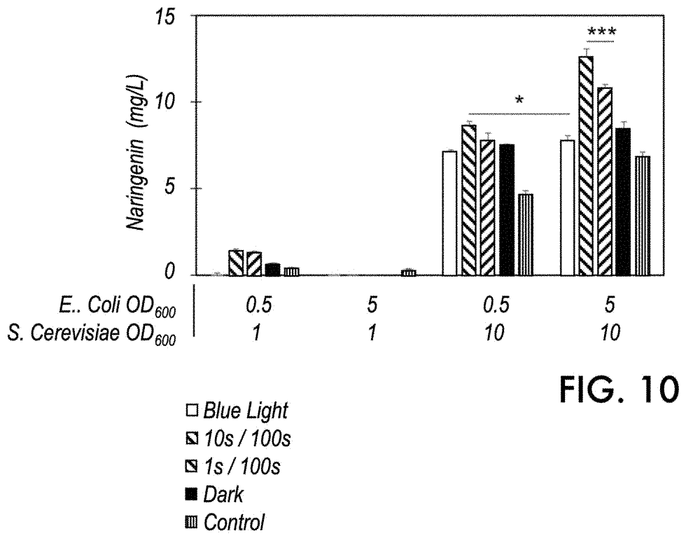

Fermentations initiated from inoculums of various sizes and proportions and conducted under different light conditions revealed that, as with isobutyl acetate, naringenin production depends on both variables. The highest naringenin titers (12.6±0.5 mg/L) are obtained at intermediate levels of light exposure, this time using a 10% (10 s ON/100 s) duty cycle and the largest inoculum sizes tested (see FIG. 10).

This titer is not only 84% higher than what a light-insensitive control strain of E. coli produces (6.8±0.2 mg/L), but it is also 64% higher than what is obtained with the light-sensitive strain under full light (7.7±0.3 mg/L) and 50% higher than what is produced by the same strain under darkness (8.4±0.4 mg/L).

This suggests that the populations of E. coli and yeast obtained in co-cultures of the light-sensitive strain exposed to intermediate levels of light are more favorable for naringenin production, and that these populations can be achieved by the optogenetic regulation afforded by OptoTA. The similar or lower levels of production of the negative controls (conducted in the dark with a light-insensitive strain) compared to the full light controls indicate that differences in naringenin titers under various light duty cycles reflect real improvements in production due to optogenetic growth control, and are not due to naringenin photobleaching in full light conditions, although light sensitivity of the product could be influencing the final titers.

Production is still influenced by initial inoculums, with the most productive yielding 46% higher production than that of a co-culture initiated with a 10-fold lower E. coli inoculum and kept under the same light conditions (8.7±0.2 mg/L), indicating that higher levels of the first module are beneficial but must be carefully regulated. Additionally, production is completely abolished when the S. cerevisiae inoculum is reduced 10-fold, emphasizing the importance of having an abundance of the second yeast module. Nevertheless, these results confirm that the additional degree of freedom afforded by optogenetic controls of co-cultures is applicable to multiple biosynthetic pathways.

Thus, tunable control of microbial consortia using light has been established, which addresses the longstanding challenge of dynamically regulating population dynamics for optimal co-culture fermentation performance. The disclosed genetic circuit is not tethered to the properties of a specific metabolic pathway, allowing it to function independently of strain modifications (such as gene overexpression or deletions) catered to specific biosynthetic pathways, and preferably requiring only that the endogenous mazEF genes are deleted. This advantage was shown by using OptoTA to control co-culture production of two chemicals derived from very different metabolic pathways: isobutyl acetate (central carbon metabolism, branched-chain alcohol biosynthesis) and naringenin (aromatic amino acid biosynthesis). It is readily understood that other biosynethic pathways can be similarly divided up and implemented with no undue experimentation required.

Dividing metabolic pathways into submodules within different strains allows for optimization of individual modules without genetically or metabolically overburdening a single strain. By using E. coli to produce isobutanol and tyrosine at high levels, we avoid extensive genetic modification of S. cerevisiae, which endogenously regulates production of both molecules. In doing so, we can focus engineering of the yeast module on overproducing other precursors—acetyl-CoA for isobutyl acetate, malonyl-CoA for naringenin, which compete for pyruvate with the upstream modules—as well as downstream biosynthesis of the final products.

Production could likely be improved by increasing the copy number of bottleneck enzymes, deleting competing endogenous enzymes, increasing transport of intermediates between modules, and/or developing a media formulation that better balances the needs of both species.

OptoTA functions by regulating the expression levels of the mazEF toxin-antitoxin system, which is endogenous to E. coli. MazEF-like paralogs have been discovered in other bacterial species, raising the possibility that OptoTA could be directly used in other bacterial species to impart optogenetic growth control, while likely exhibiting less cross-talk with endogenous systems. Alternatively, the architecture of OptoTA could feasibly be applied to control other toxin-antitoxin systems, which are ubiquitous in nature, enabling optogenetic regulation of cell growth in diverse bacterial genera such as Corynebacterium, Bacillus, Pseudomonas, or Lactococcus using different toxin-antitoxin pairs. Building redundancies by simultaneously using multiple TA systems (including with different mechanisms of action) in a single strain could help attain more stringent growth control and attenuate the variability observed in different media. Light-dependent growth of other commonly used model organisms, each with its own advantages, could make different types of light-controlled microbial consortia viable for a variety of biotechnological applications.

The specific implementation of OptoTA uses single-input (blue light) control over consortia in which the growth rates of two species are heavily stratified, such as the E. coli-S. cerevisiae co-culture. This strategy is extendable to situations in which fermentation conditions (pH, temperature, oxygenation, media) are disadvantageous to one organism. It could potentially also be used for co-cultures featuring two different strains of the same species but containing genetic modifications that diverge their growth rates. Fermentations for chemical production in lab-scale bioreactors have been optogenetically controlled using pDawn. However, at larger scales, in which light penetration may become limiting, YF1 mutants with increased light sensitivity could be incorporated to achieve the required light stimulation. OptoTA establishes light control over the faster growing strain irrespective of process conditions or genetic background, allowing for flexible implementation of co-cultures for a wide variety of conditions and applications.

However, orthogonal optogenetic growth regulators that respond to stimuli other than blue light could be developed and used alongside OptoTA to effectively engineer and control more complex microbial co-cultures. Additional optogenetic systems that respond to darkness could be employed to allow opposing growth in co-cultured species in the presence or absence of light.

Alternatively, optogenetic systems such as those based on phytochromes and cyanobacteriochromes, which respond to green, red, and far-red light, could enable decoupled control of multiple species using different wavelengths. Such polychromatic multi-input growth controls could be used to dynamically regulate population of consortia with more complex functions, including those that feature strains or species with similar growth profiles or contain more than two members.

The improved control of optogenetics over co-culture fermentations raises a unique optimization problem: how to adjust the timing and duration of light pulses to optimize populations to maximize production from co-cultures. Previous studies have investigated in silico approaches to analyze (and potentially engineer) interpopulation dynamics in microbial communities, as well as computer-assisted control of optogenetic inputs to stabilize gene expression profiles toward desired set points. Moving forward, kinetic models could be designed based on growth data (species, light control) and pathway information (precursor supply, metabolic branch points) to predict highly productive population regimes for different products a priori, as well as to implement control strategies to keep a co-culture within these desired regimes via well-timed light pulses. Populations could be distinguished using fluorescent markers, or other quickly measurable outputs such as pH, oxygenation, and temperature, to provide live feedback to the optogenetic controller. Such “cybergenetic” approaches could substantially increase the throughput of process optimization while alleviating the burden of user control over implementing complex light schedules.

Optogenetic regulation of populations in microbial communities constitutes a new paradigm that could impact the fields of metabolic engineering, fermentation technology, and beyond. The unique advantages that light provides can make it possible to identify and maintain optimal microbial populations throughout fermentation for maximal chemical production. Optogenetics could thus help resolve the long-standing challenge of population control in co-culture fermentations and help realize the full potential of improving biosynthesis of complex chemical products by microbial division of labor. The disclosed approach offers a blueprint to establish optogenetic controls of microbial communities in different settings such as in the areas of bioremediation, agriculture and food technology, and microbiome research.

What is claimed is:

1. An engineered strain of a microorganism, comprising:
a genetic circuit configured to express a gene that enables growth when exposed to a light condition, and express a gene that represses growth when not exposed to the light condition,
wherein the genetic circuit comprises:
a first polypeptide sequence encoding a repressor, such as the λ phage repressor cI, under a first promoter, the first promoter being a light-responsive promoter;
a second polypeptide sequence encoding either a toxin or an antitoxin, under a second promoter, the second promoter being controlled by the repressor encoded by the first sequence, such as the λ promoter; and
a third polypeptide sequence encoding the other of the toxin or the antitoxin, under an additional first promoter.

2. The engineered strain of a microorganism according to claim 1, wherein the genetic circuit is configured to express a bacteria toxin only when exposed to the light condition and a bacteria antitoxin when not exposed to the light condition or when exposed to a different wavelength of light.

3. The engineered strain of a microorganism according to claim 2, wherein the microorganism is a strain of E. coli.

4. The engineered strain of a microorganism according to claim 3, wherein the bacteria toxin is MazF, and the bacteria antitoxin is MazE.

5. The engineered strain of a microorganism according to claim 1, wherein the light condition is irradiation by a visible and/or infrared wavelength of light.

6. The engineered strain of a microorganism according to claim 1, wherein the genetic circuit comprises pDawn and pDusk optogenetic circuits.

7. The engineered strain of a microorganism according to claim 1, wherein the first promoter is P_{FixK2}, and the second promoter is P_{R}.

8. A method for regulating engineered microbial consortia, comprising:
providing a plurality of microorganisms, where at least one of the plurality of microorganisms comprises a genetic circuit utilizing an optogenetic gene expression system to control an organism's growth rate using a specific wavelength of light or darkness, and
co-culturing the plurality of microorganisms in a light-controlled fermentation.

9. The method according to claim 8,
wherein at least a first organism of the plurality of microorganisms comprises a biosynthetic pathway that produces an intermediate metabolite and at least a second organism of the plurality of microorganisms comprises a biosynthetic pathway that converts the intermediate metabolite into a chemical of interest, and
wherein co-culturing the plurality of microorganisms in light-controlled fermentation comprises adjusting light conditions to control the co-culture population composition and production of the chemical of interest.

10. The method according to claim 9, wherein the chemical of interest is an ester or flavonoid.

11. The method according to claim 9, further comprising, in at least one of the plurality of microorganisms, transforming the microorganism with a first plasmid or vector that integrates into a chromosome, which contains the genetic circuit that uses an optogenetic gene expression system to control an organism's growth rate using a specific wavelength of light or darkness.

12. The method according to claim 9, further comprising, in at least one of the plurality of microorganisms, transforming the microorganism with a second plasmid or vector that integrates into a chromosome, which contains the biosynthetic pathway that produces the intermediate metabolite.

13. The method according to claim 9, further comprising, in at least one of the plurality of microorganisms, transforming the microorganism with a third plasmid or vector that integrates into a chromosome, which contains a biosynthetic pathway that converts an intermediate metabolite into the chemical of interest.

14. The method according to claim 9, wherein the intermediate metabolite is an alcohol or aromatic amino acid.

15. The method according to claim 8, wherein the plurality of microorganisms comprises one or more strains of E. coli, S. cerevisiae, P. putida, or a combination thereof.

16. The method according to claim 8, wherein co-culturing the plurality of microorganisms in light-controlled fermentation comprises illuminating the co-cultured fermentations with varying light schedules.

17. The method according to claim 8, wherein the plurality of microorganisms comprises a bacteria species and a yeast species, and has a bacteria-to-yeast ratio<1.

18. The method according to claim 8, wherein the plurality of microorganisms comprises a bacteria species and a yeast species, and has a bacteria-to-yeast ratio=1.

19. The method according to claim 8, wherein the plurality of microorganisms comprises a bacteria species and a yeast species, and has a bacteria-to-yeast ratio>1.

20. The method according to claim 8, wherein co-culturing utilizes a starting OD₆₀₀ of bacteria≤5.

21. The method according to claim 8, wherein co-culturing utilizes a starting OD₆₀₀ of bacteria >1×10⁻³.

22. The method according to claim 8, wherein co-culturing utilizes a starting OD₆₀₀ of yeast of≥0.1.

23. The method according to claim 8, wherein co-culturing utilizes and a starting OD₆₀₀ of yeast of ≤10.
